(19) United States
(12) Patent Application Publication
(10) Pub. No.: US 2013/0095051 A1
(43) Pub. Date: Apr. 18, 2013

(54) AVERMECTIN/METRONIDAZOLE COMPOSITIONS FOR TREATING AFFLICTIONS OF THE SKIN, E.G., ROSACEA

(71) Applicant: Galderma S.A., Cham (CH)

(72) Inventors: Alexandre KAOUKHOV, Juan Les Pins (FR); Colette PERNIN, Nice (FR)

(73) Assignee: GALDERMA S.A., Cham (CH)

(21) Appl. No.: 13/707,140

(22) Filed: Dec. 6, 2012

Related U.S. Application Data
(63) Continuation of application No. 13/198,599, filed on Aug. 4, 2011.
Application No. 13/198,599 is a continuation of application No. 12/834,095, filed on Jul. 12, 2010, now abandoned.
Application No. 12/834,095 is a continuation of application No. 12/000,182, filed on Dec. 10, 2007, now abandoned.
Application No. 12/000,182 is a continuation of application No. PCT/FR2006/001301, filed on Jun. 8, 2006.
Application No. PCT/FR2006/001301 is a continuation of application No. 13/039,882, filed on Mar. 3, 2011.
Application No. 13/039,882 is a continuation of application No. 12/394,234, filed on Feb. 27, 2009, now abandoned.
Application No. 12/000,182 is a continuation of application No. PCT/FR2006/001301, filed on Jun. 8, 2006.

(30) Foreign Application Priority Data
Jun. 10, 2005 (FR) 05/05918

(51) Int. Cl. A61K 31/7048; A61K 31/4164

(52) U.S. Cl. CPC A61K 31/7048; A61K 31/4164
USPC 424/59; 514/30

(57) ABSTRACT

Pharmaceutical/dermatological compositions containing at least one avermectin compound, e.g., ivermectin and metronidazole or salt, ester or derivative thereof, are useful for treating afflictions of the skin, especially rosacea.

### CROSS-REFERENCE TO EARLIER APPLICATIONS

This application is a continuation of U.S. application Ser. No. 13/198,599, filed Aug. 4, 2011, and a continuation of U.S. application Ser. No. 13/039,882, filed Mar. 3, 2011. U.S. application Ser. No. 13/198,599 is a continuation of U.S. application Ser. No. 12/834,095, filed Jul. 12, 2010, now abandoned, which is a continuation of U.S. application Ser. No. 12/000,182, filed Dec. 10, 2007, now abandoned, which is a continuation of PCT/FR 2006/001301, filed Jun. 8, 2006, and designating the United States (published in the French language on Dec. 14, 2006 as WO 2006/131653 A1; the title and abstract were published in English), which claims priority under 35 U.S.C. §119 of FR 05/05918, filed Jun. 10, 2005. U.S. application Ser. No. 13/039,882 is a continuation of U.S. application Ser. No. 12/394,234, filed Feb. 27, 2009, now abandoned, which is a divisional of U.S. application Ser. No. 12/000,182, filed Dec. 10, 2007, now abandoned, which is a continuation of PCT/FR 2006/001301, filed Jun. 8, 2006, and designating the United States (published in the French language on Dec. 14, 2006 as WO 2006/131653 A1; the title and abstract were published in English), which claims priority under 35 U.S.C. §119 of FR 05/05918, filed Jun. 10, 2005.

### BACKGROUND OF THE INVENTION

1. Technical Field of the Invention

The present invention relates to pharmaceutical compositions, and especially dermatological compositions, for treating skin conditions and afflictions, and especially for treating rosacea (formerly known as acne rosacea).

In particular, the present invention relates to pharmaceutical compositions, especially dermatological compositions, comprising, formulated into a physiologically acceptable medium, at least one compound of the avermectin family and metronidazole.

2. Description of Background and/or Related and/or Prior Art

Rosacea is a chronic inflammatory dermatitis that mainly affects the median part of the face and the eyelids of certain adults. It is characterized by telangiectatic erythema, dryness of the skin, papules and pustules.

Conventionally, rosacea develops in adults from the ages of 30 to 50; it more frequently affects women, although the condition is generally more severe in men.

Despite its former name, acne rosacea is not a condition of the pilosebaceous follicles like juvenile acne, but a primitively vascular condition whose inflammatory stage lacks the cysts and comedones characteristic of common acne.

The aetiology of rosacea is still poorly understood, although many theories have been advanced. The most common hypothesis is based on the characteristic presence of the parasite Demodex folliculorum in the case of patients suffering from rosacea. This organism is absent in the other forms of acne such as common acne. Other factors have been described as possibly contributing towards the development of rosacea, such as hormonal factors and especially endocrine factors, climatic and immunological factors, and bacterial factors via the presence of Helicobacter pylori, a bacterium associated with gastrointestinal disorders.

Rosacea develops in four stages over several years, in spasms aggravated by variations in temperature, alcohol, spices, exposure to sunlight and emotions. The various stages of the disease are the following:

Stage 1: stage of erythema episodes. The patients have erythrosis spasms due to the sudden dilation of the arterioles of the face, which then take on a congestive, red appearance. These spasms are caused by the emotions, meals and temperature changes.

Stage 2: stage of couperosis, i.e., of permanent erythema with telangiectasia. Certain patients also have oedema on the cheeks and the forehead.

Stage 3: inflammatory stage with appearance of inflammatory papules and pustules, but without affecting the sebaceous follicles and thus with absence of cysts and comedones.

Stage 4: rhinophyma stage. This late phase essentially affects men. The patients have a bumpy, voluminous red nose with sebaceous hyperplasia and fibrous reordering of the connective tissue.

Conventionally, rosacea is treated orally or topically with antibiotics such as tetracyclines, erythromycin or clindamycin, but also with vitamin A, salicylic acid, anti-fungal agents, steroids, anti-infectious agents such as benzoyl peroxide, or with isotretinoin in severe cases or even with metronidazole (an anti-bacterial agent).

Metronidazole is known in the prior art for its anti-parasitic, anti-protozoan and anti-bacterial properties. It is especially used for treating Helicobacter pylori infections. It is also prescribed in the treatment of rosacea, for its advantageous properties on the inflammatory lesions of rosacea, specifically on papules and pustules. Metronidazole exerts selective toxicity towards anaerobic microorganisms and also hypoxic cells. On the latter, metronidazole is reduced to various derivatives that are capable of changing the structure of their DNA.

U.S. Pat. No. 5,952,372 also describes a method for treating rosacea using ivermectin orally or topically in order to reduce and eliminate the parasite Demodex folliculorum present on the skin of patients.

Ivermectin belongs to the avermectin family, a group of macrocyclic lactones produced by the bacterium Streptomyces avermitilis (Reynolds JEF (Ed) (1993) Martindale. The Extra Pharmacopoeia., 29th Edition. Pharmaceutical Press, London).

The avermectins especially include ivermectin, invermectin, avermectin, abamectin, doramectin, eprinomectin and selamectin.

Ivermectin is known in the prior art for its anti-parasitic and anthelmintic properties. The anti-parasitic activity is thought to be due to the opening of a chlorine channel in the membrane of the neurons of the parasite under the effect of an increased release of the neuromediator GABA (gamma-aminobutyric acid), inducing neuromuscular paralysis that may lead to the death of certain parasites. Ivermectin also interacts with other chlorine channels, especially those dependent on the neuromediator GABA (gamma-aminobutyric acid).

Ivermectin is conventionally administered in the dermatological treatment of endoparasitic manifestations such as onchocerciasis and myiasis. U.S. Pat. No. 6,133,310 describes the use of ivermectin in the treatment of rosacea in order to reduce and eliminate the parasite Demodex folliculorum present on the skin of patients.

However, these treatments have drawbacks such as irritation and intolerance phenomena, especially when they are administered for a prolonged period. On the other hand, these treatments are only suppressive and not curative, acting especially on the pustulous spasms occurring during the inflammatory stage.

Considering the chronic nature of rosacea, the ideal treatment requires prolonged use, in a safe and effective manner. Taking the foregoing into account, need thus exists for compositions that show improved efficacy in the treatment of rosacea and that do not have the side effects described in the prior art.

### SUMMARY OF THE INVENTION

Accordingly, the present invention features pharmaceutical compositions, especially dermatological compositions, comprising, formulated into a physiologically acceptable medium, at least one compound of the avermectin family and metronidazole.

The term “physiologically acceptable medium” means any medium that is compatible with the skin, mucous membranes and/or the integuments.

The metronidazole according to the invention may be used in unmodified form, or alternatively in the form of a salt with an acid or a pharmaceutically acceptable base, or else in the form of an ester or of a derivative. The term “esters” means metronidazole acetate or metronidazole benzoate. The term “derivatives” means compounds that differ from azelaic acid by substitution, addition or removal of one or more chemical groups and that have substantially the same activity.

Thus, this invention features pharmaceutical compositions, especially dermatological compositions, comprising, formulated into a physiologically acceptable medium, at least one compound of the avermectin family, and at least one compound selected from among metronidazole, and salts, esters and derivatives thereof.

The present invention preferentially features pharmaceutical compositions, especially dermatological compositions, comprising, formulated into a physiologically acceptable medium, at least ivermectin and metronidazole.

This invention also features compositions formulated as medicaments for preventing and/or treating a skin condition.

Such compositions are especially for topical application.

The invention and the advantages resulting therefrom will become more apparent from the description which follows.

### DETAILED DESCRIPTION OF BEST MODE AND SPECIFIC/PREFERRED EMBODIMENTS OF THE INVENTION

The compounds of the avermectin family according to the present invention especially include invermectin, ivermectin, avermectin, abamectin, doramectin, eprinomectin and selamectin. The compound of the avermectin family is preferentially ivermectin.

In the compositions according to the invention, the said compound of the avermectin family is present in concentrations of from 0.001% to 10% by weight and preferably from 0.01% to 5% by weight relative to the total weight of the composition.

In the compositions according to the invention, the metronidazole, salts, esters and/or derivatives thereof is/are present in concentrations of from 0.0001% to 20% by weight and preferably from 0.01% to 10% by weight and particularly preferably from 0.1% to 2% by weight relative to the total weight of the composition.

Herein, unless otherwise specified, it is understood that when concentration ranges are given, they include the upper and lower limits of the said range.

Advantageously, the compositions of the invention comprise, other than the at least one compound of the avermectin family and metronidazole, at least one other therapeutic agent capable of increasing the efficacy of the treatment. Exemplary such agents include antibiotics, anti-bacterial agents, anti-viral agents, anti-parasitic agents, anti-fungal agents, anaesthetics, analgesics, anti-allergic agents, retinoids, free-radical scavengers, anti-pruritic agents, keratolytic agents, anti-seborrhoeic agents, anti-histamines, sulfides, and immunosuppressant or anti-proliferative products, or a mixture thereof.

The compositions according to the invention may also comprise any adjuvant usually employed in cosmetics and dermatology that is compatible with the said compound of the avermectin family and metronidazole. Especially exemplary are chelating agents, antioxidants, sunscreens, preservatives, fillers, electrolytes, humectants, dyes, common mineral or organic acids or bases, fragrances, essential oils, cosmetic active agents, moisturizers, vitamins, essential fatty acids, sphingolipids, self-tanning compounds, calmatives and skin-protecting agents, pro-penetrating agents and gelling agents, or a mixture thereof. These adjuvants, and the concentrations thereof, should be such that they do not adversely affect the advantageous properties of the mixture according to the invention. These additives may be present in the composition in a proportion of from 0% to 20% by weight and preferably from 1% to 10% by weight relative to the total weight of the composition.

Exemplary preservatives include benzalkonium chloride, phenoxyethanol, benzyl alcohol, diazolidinylurea and parabens, or mixtures thereof.

Humectants that are exemplary include glycerol and sorbitol.

Exemplary chelating agents include ethylenediaminetetraacetic acid (EDTA) and also derivatives or salts thereof, dihydroxyethylglycine, citric acid and tartaric acid, or mixtures thereof.

Pro-penetrating agents that are exemplary include propylene glycol, dipropylene glycol, propylene glycol dipelargonate, lauryl glycol and ethoxydiglycol.

The compositions according to the invention are useful, whether in a regime or regimen, for treating and/or preventing rosacea.

According to a first embodiment of the invention, the subject compositions are useful for formulating medicaments for treating the skin and preferably for treating rosacea, common acne and seborrhoeic dermatitis and particularly preferably for treating rosacea.

The present invention also features the use of at least one compound of the avermectin family and of at least one compound selected from metronidazole, salts, esters and/or derivatives thereof for the formulation of pharmaceutical compositions, and especially dermatological compositions, for preventing and/or treating a skin condition.

The compositions according to the invention are pharmaceutical compositions, and especially dermatological compositions, which may be in any galenical form conventionally used for topical application and especially in the form of aqueous gels, and aqueous or aqueous-alcoholic solutions. By addition of a fatty or oily phase, it may also be in the form of dispersions of the lotion or serum type, emulsions of liquid or semi-liquid consistency of the milk type obtained by dispersing a fatty phase in an aqueous phase (O/W) or conversely (W/O), or suspensions or emulsions of soft, semi-liquid or solid consistency of the cream, gel or ointment type, or alternatively multiple emulsions (W/O/W or O/W/O), microemulsions, microcapsules, microparticles or vesicular dispersions of ionic and/or nonionic type, or wax/aqueous phase dispersions. These compositions are formulated according to the usual methods.

When the composition is in emulsion form, the proportion of the oily phase of the emulsion may range, for example, from 5% to 80% by weight and preferably from 5% to 50% by weight relative to the total weight of the composition. The oils, emulsifiers and co-emulsifiers used in the composition in emulsion form are selected from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

As fatty substances that may be used in the invention, exemplary are oils and especially mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols such as cetyl alcohol, fatty acids, waxes and gums, in particular silicone gums, may also be used as fatty substances.

As emulsifiers and co-emulsifiers according to the invention, exemplary are fatty acid esters of polyethylene glycol such as PEG-100 stearate, PEG-50 stearate and PEG-40 stearate; fatty acid esters of polyols such as glyceryl stearate, sorbitan tristearate and the oxyethylenated sorbitan stearates available under the trademark Tween 20 or Tween 60, for example; and mixtures thereof.

Examples of gelling agents include the polyacrylamide family such as the sodium acryloyldimethyltaurate copolymer/isohexadecane/polysorbate 80 mixture marketed under the trademark Simulgel™ 600 by SEPPIC, the polyacrylamide/C13-14 isoparaffin/Laureth-7 mixture, for instance the product marketed under the trademark Sepigel 305™ by SEPPIC, the family of acrylic polymers coupled to hydrophobic chains, such as the PEG-150/decyl/SMDI copolymer marketed under the trademark Aculyn 44™ (polycondensate comprising at least, as components, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%)), and the family of modified starches such as the modified potato starch marketed under the trademark Structure Solanace™, or mixtures thereof.

The preferred gelling agents are derived from the polyacrylamide family, such as Simulgel 600™ or Sepigel 305™, or mixtures thereof.

The gelling agent as described above may be used in a concentration ranging from 0.1% to 15% and preferably from 0.5% to 5%.

Each patent, patent application, publication, text and literature article/report cited or indicated herein is hereby expressly incorporated by reference in its entirety. While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed:

1. A pharmaceutical topically applicable composition useful for treating rosacea, comprising anti-rosacea effective amounts of ivermectin and metronidazole, formulated into a topically applicable, physiologically acceptable medium therefor.

2. The pharmaceutical composition as defined by claim 1, comprising from 0.001% to 10% by weight of ivermectin relative to the total weight thereof.

3. The pharmaceutical composition as defined by claim 1, comprising from 0.0001% to 20% by weight of metronidazole relative to the total weight of the composition.

4. The pharmaceutical composition as defined by claim 1, further comprising at least one additive selected from the group consisting of chelating agents, antioxidants, sunscreens, preservatives, fillers, electrolytes, humectants, dyes, mineral acids, organic acids, mineral bases, organic bases, fragrances, essential oils, cosmetic active agents, moisturizers, vitamins, essential fatty acids, sphingolipids, self-tanning compounds, calmatives, skin-protecting agents, pro-penetrating agents, gelling agents, and mixtures thereof.

5. The pharmaceutical composition as defined by claim 1, comprising from 0.01% to 5% by weight of ivermectin relative to the total weight thereof and from 0.01% to 2% by weight of metronidazole relative to the total weight thereof.

6. A pharmaceutical topically applicable composition useful for treating rosacea, comprising anti-rosacea effective amounts of ivermectin and metronidazole, formulated into a topically applicable, physiologically acceptable medium therefor, said ivermectin and metronidazole being the only therapeutic active anti-rosacea agents in the composition.

7. The pharmaceutical composition as defined by claim 6, comprising from 0.01% to 5% by weight of ivermectin relative to the total weight thereof and from 0.01% to 2% by weight of metronidazole relative to the total weight thereof.

8. A pharmaceutical topically applicable composition useful for treating rosacea, consisting of anti-rosacea effective amounts of ivermectin and metronidazole, formulated into a topically applicable, physiologically acceptable medium therefor.

9. The pharmaceutical composition as defined by claim 8, ivermectin being from 0.01% to 5% by weight relative to the total weight thereof and metronidazole being from 0.01% to 2% by weight relative to the total weight thereof.

10. A pharmaceutical topically applicable composition useful for treating rosacea, consisting of anti-rosacea effective amounts of ivermectin and metronidazole, formulated into a topically applicable, physiologically acceptable medium therefor, said medium consisting of (a) at least one member selected from the group consisting of water, alcohols, oils, fatty substances and waxes; and (b) at least one additive selected from the group consisting of chelating agents, antioxidants, sunscreens, preservatives, fillers, electrolytes, humectants, dyes, mineral acids, mineral bases, organic acids, organic bases, fragrances, essential oils, moisturizers, vitamins, essential fatty acids, sphingolipids, self-tanning compounds, calmatives, skin-protecting agents, pro-penetrating agents, gelling agents, emulsifiers, co-emulsifiers, and mixtures thereof.

11. The pharmaceutical composition as defined by claim 10, ivermectin being from 0.01% to 5% by weight relative to the total weight thereof and metronidazole being from 0.01% to 2% by weight relative to the total weight thereof.

12. The pharmaceutical composition as defined by claim 5, formulated as an emulsion, a gel, or a cream.

13. A method for treating rosacea, comprising topically applying onto the afflicted skin area of an individual in need of such treatment, a pharmaceutical/dermatological topically applicable composition comprising anti-rosacea effective amounts of ivermectin and metronidazole, formulated into a topically applicable, physiologically acceptable medium therefor.

14. The method as defined by claim 13, wherein said composition comprises from 0.001% to 10% by weight of ivermectin and from 0.0001% to 20% by weight of metronidazole relative to the total weight of the composition.

15. The method as defined by claim 13, wherein said composition further comprises at least one additive selected from the group consisting of chelating agents, antioxidants, sunscreens, preservatives, fillers, electrolytes, humectants, dyes, mineral acids, organic acids, mineral bases, organic bases, fragrances, essential oils, cosmetic active agents, moisturizers, vitamins, essential fatty acids, sphingolipids, self-tanning compounds, calmatives, skin-protecting agents, pro-penetrating agents, gelling agents, and mixtures thereof.

16. The method as defined by claim 13, wherein said composition comprises from 0.01% to 5% by weight of ivermectin relative to the total weight thereof and from 0.01% to 2% by weight of metronidazole relative to the total weight thereof.

17. A method for treating rosacea, comprising topically applying onto the afflicted skin area of an individual in need of such treatment, a pharmaceutical topically applicable composition comprising anti-rosacea effective amounts of ivermectin and metronidazole, formulated into a topically applicable, physiologically acceptable medium therefor, said ivermectin and metronidazole being the only therapeutic active anti-rosacea agents in the composition.

18. The method as defined by claim 17, wherein said composition comprises from 0.01% to 5% by weight of ivermectin relative to the total weight thereof and from 0.01% to 2% by weight of metronidazole relative to the total weight thereof.

19. A method for treating rosacea consisting of topically applying onto the afflicted skin area of an individual in need of such treatment, a pharmaceutical/dermatological topically applicable composition consisting of anti-rosacea effective amounts of ivermectin and metronidazole, formulated into a topically applicable, physiologically acceptable medium therefor.

20. The method as defined by claim 19, wherein, in said composition, ivermectin is present in an amount of from 0.01% to 5% by weight relative to the total weight thereof and metronidazole is present in an amount of from 0.01% to 2% by weight relative to the total weight thereof.

21. A method for treating rosacea, consisting of topically applying onto the afflicted skin area of an individual in need of such treatment, a pharmaceutical/dermatological topically applicable composition consisting of anti-rosacea effective amounts of ivermectin and metronidazole, formulated into a topically applicable, physiologically acceptable medium therefor, said medium consisting of (a) at least one member selected from the group consisting of water, alcohols, oils, fatty substances and waxes; and (b) at least one additive selected from the group consisting of chelating agents, antioxidants, sunscreens, preservatives, fillers, electrolytes, humectants, dyes, mineral acids, mineral bases, organic acids, organic bases, fragrances, essential oils, moisturizers, vitamins, essential fatty acids, sphingolipids, self-tanning compounds, calmatives, skin-protecting agents, pro-penetrating agents, gelling agents, emulsifiers, co-emulsifiers, and mixtures thereof.

22. The method as defined by claim 21, wherein, in said composition, ivermectin is present in an amount of from 0.01% to 5% by weight relative to the total weight thereof and metronidazole is present in an amount of from 0.01% to 2% by weight relative to the total weight thereof.

23. The method as defined by claim 16, wherein said composition is formulated as an emulsion, a gel, or a cream.
